(19) United States
(12) Patent Application Publication
(10) Pub. No.: US 2025/0359569 A1
(43) Pub. Date: Nov. 27, 2025

(54) COMPOSITION COMPRISING DRY EXTRACT OF LYOPHILISED INTESTINAL CONTENT OF ADULT CHICKEN, RELATIVE USE AS FOOD SUPPLEMENT AND USE FOR STIMULATING THE IMMUNE SYSTEM

(71) Applicants: UNIVERSITA' DEGLI STUDI DI CAMERINO, Camerino (MC) (IT); ORBIOTEK SÀRL, Losanna (CH)

(72) Inventors: Giacomo Rossi, Camerino (MC) (IT); Lucia Biagini, Camerino (MC) (IT); Livio Galosi, Camerino (MC) (IT)

(21) Appl. No.: 18/569,859

(22) PCT Filed: Jun. 17, 2022
(86) PCT No.: PCT/IB2022/055647

§ 371 Date: Dec. 13, 2023

(30) Foreign Application Priority Data
Jun. 18, 2021 (IT) 102021000016055

(51) Int. Cl. A23K 10/18; A23K 40/00; A23K 50/75; A61K 39/00; A61K 39/255

(52) U.S. Cl. CPC A23K 10/18; A23K 40/00; A23K 50/75; A61K 39/255; A61K 2039/54; A61K 2039/552

(57) ABSTRACT

The present invention relates to a composition comprising dry extract of intestinal content of adult chickens lyophilised by spray technique and tyndallised in batch mode, the use thereof as a food supplement and the use thereof to stimulate the immune system.

### CROSS-REFERENCE TO RELATED APPLICATIONS

This patent application claims priority from Italian Patent Application No. 102021000016055 filed on Jun. 18, 2021, the entire disclosure of which is incorporated herein by reference.

### TECHNICAL FIELD OF THE INVENTION

The present invention relates to a composition comprising dry extract of intestinal content of adult chicken lyophilised by spray technique and tyndallised in batch mode, to the relative use as a food supplement and to the relative use as an immune system stimulator, particularly for chickens.

### STATE OF THE ART

The use of sub-therapeutic doses of antibiotics in animal feed has been a widespread practice in recent decades. In the poultry industry, it has been used mainly for auxinic purposes, i.e. with the aim of promoting the weight gain of animals. However, concerns have been raised about the consequences of this overuse, especially with regard to the issue of antibiotic resistance. The danger posed by the acquisition of antibiotic resistance in microorganisms is directed not only to animal health, but above all to human health, as the possibility of the transfer of antibiotic resistance genes also to the microorganisms that are pathogenic to humans is real. The progressive reduction in the use of antimicrobials in the zootechnical field has led to the need to look for alternative substances for disease control and prevention. Hence the interest in the role of the gut microbiota, whose interactive capacity with its host, influencing its morphology and intestinal physiology, as well as actively stimulating its immune system, has been demonstrated by several studies. For this reason, it was thought to act from the outside to try to modulate the gut microbiota in various ways and achieve desired beneficial effects (Pan and Yu, 2014). One path that has proved promising has been that of the “biotics” (prebiotics, probiotics, synbiotics, postbiotics and para-probiotics) which have been studied precisely for the ability of modulating the gut microbiota and consequently of influencing the development, the physiology and the immunological response of the gastrointestinal tract. All “biotics” act by managing to increase, even potentially, the resistance of the host against infections thanks to their interaction with the intestinal wall and the lymphoid tissues that make up the GALT.

Probiotics are defined by FAO and WHO as “living micro-organisms that, when administered in an adequate quantity, bring a benefit to the health of the host”. There are a large number of studies in the bibliography describing the beneficial effects deriving from the use of probiotics in the poultry field, mainly in addition to food or drinking water. IT has been described how the administration of probiotics ensures better nutrient use and increased growth performance of the animals (Ignatova et al., 2009; Kabir et al., 2004; Khaksefidi and Ghoorchi, 2006; Nayebpor et al., 2007; Sen et al., 2012; Talebi et al., 2008; Edens et al. 2003; Angel et al., 2005). In addition, the use of probiotics allows having intestinal colonisation with specific non-pathogenic bacteria and obtaining a protection against pathogens (De Oliveira, 2014). In this respect, several studies have demonstrated, following probiotic administration, an increased resistance against bacterial, viral and protozoan pathogens such as Salmonella spp., Clostridium spp., Eimeria spp. (Pender et al., 2016; Dalloul e Lillehoj, 2006; Lee et al., 2007; Teague et al., 2017; Jayaraman et al., 2013). Another key element in favour of using probiotics as modulators of the gut microbiota is the influence on the immune system in both the innate and acquired components (Farnell et al., 2006; Higgins et al., 2007; Brisbin et al., 2011; Stringfellow et al., 2011; Bai et al., 2013; Karaffová et al., 2017; Pender et al., 2017).

Probiotics, together with prebiotics, are certainly among the most studied substances as alternatives to antibiotics. However, more recently, the interest has been focused on the use of other probiotic-correlated components. The terms paraprobiotics and postbiotics emerged from the idea that bacterial viability is not an essential element for the beneficial effects to be obtained (Aguilar-Toalá et al., 2018). The term paraprobiotics was proposed by Taverniti and Guglielmetti (2011) and refers to intact or lysed non-viable microbial cells, which when administered in sufficient quantity confer beneficial effects on the consumer (Taverniti and Guglielmetti, 2011). Instead, postbiotics refer to soluble factors secreted by live bacteria or released after bacterial lysis and include: short-chain fatty acids, enzymes, peptides, endo- and exo-polysaccharides, cell surface proteins, vitamins and organic acids and fragments of the microorganisms themselves (Konstantinov et al., 2013; Tsilingiri & Rescigno, 2013).

Although the beneficial effects of probiotics have been proven, the rationale for using non-viable microorganisms stems from the idea that there may be greater advantages in terms of safety by reducing the risk of systemic infections, excessive local inflammatory response, acquisition of antibiotic resistance genes, which have been demonstrated for some probiotics (Tsilingiri et al., 2012; Taverniti and Guglielmetti, 2011). Although there is a growing interest in the use of para- and postbiotics, the literature on the subject is less extensive, especially with regard to the specific use in livestock and poultry farming field. The use of heat-inactivated bacteria has been proven to have immunomodulatory, anti-inflammatory and antimicrobial effects (Adams et al., 2010; Habil et al., 2014; Guglielmetti et al., 2008; Taverniti and Guglielmetti, 2011; Li et al., 2009; Konstantinov et al., 2008; Sakai et al., 2006). With regard to postbiotics, some studies have been carried out in recent years concerning the effects of administration on the chicken. These have demonstrated their immunomodulatory and defensive role against certain pathogens such as Clostridium perfringens or E. coli (Johnson et al., 2019; Kareem et al., 2017; Kareem et al., 2014), but also in improving production performance (Loh et al, 2010; Rosyidah et al., 2011; Kareem et al., 2016; Klemashevich et al., 2014), modulation of gut morphology and immune response in chickens subjected to temperature stress (Humam et al., 2019; Humam et al., 2021). There are several ways to achieve probiotic inactivation or to achieve postbiotic components. The main mechanisms concern physical inactivation (mechanical destruction, heat, gamma or UV treatment, freezing, sonication) or chemical inactivation (acid deactivation).

All these compounds, whether they are probiotics, prebiotics or postbiotics, are traditionally administered in water or drinking food after the chick has been housed. However, this leads to having a reduced margin of action on the prevention of chicken diseases and on the ability of modulating the gut microbiota. Indeed, under commercial conditions, the hatchery environment represents the first risk factor for the possibility of infection developing in the chick. In addition to this, there are longer or shorter periods of time elapsing between hatching and housing moments, causing considerable stress in the newborn animal, which also favours the development of infections caused by “field” or environmental pathogens present at the time of adaptation. Furthermore, it is important to emphasise that the last days before hatching (from the 18th), and the first days afterwards, represent the transition period from ovo-dependent feeding to the beginning of spontaneous feeding through the intake of the feed given in the hatchery. These are the most critical days for the chick's survival, in fact the birds are born with an immature gastrointestinal tract and rapid morphological, biochemical and cellular changes take place immediately after birth that allow the absorption of ingested nutrients. In preventing these problems, the idea of using the in ovo technique was born. The in ovo administration of probiotics, postbiotics or prebiotics is intended to allow the establishment of a healthy and balanced gut microbiota even before birth, a fundamental factor in favouring the correct completion of the development of the gastrointestinal tract, as well as in implementing a defence against pathogens prior to contact with the same present in the environment at the time of housing.

The in ovo technique was originally developed for the administration of the vaccine for Marek's disease. Sharma and Burmester (1982) were the first ones to realise that the administration of the vaccine for Marek's disease in ovo could effectively be used in chicken to achieve early and effective immunity against the disease. Even today, this vaccination is administered in ovo on the 18th day of incubation, using automated injection systems. A rich bibliography has developed around the evaluation of the possibility of administering probiotics in ovo. The first studies date back to the 1990s with the work carried out by Cox et al. (1992), the progenitor of the topic, many others follow, differing in age, administration sites and probiotic mixtures used (De Oliveira et al., 2014; Pender et al., 2016; Teague et al., 2017; Triplett et al., 2018; Beck et al., 2019; Castaneda et al., 2020; Pender et al., 2016). In general, these studies have further confirmed the beneficial effects deriving from the use of probiotics, noting that this mode of administration is really usable as there is no influence on the hatching rate of the chicks. Although the bibliography concerning the administration of probiotics in ovo is quite extensive, there is no information concerning the administration of substances such as paraprobiotics or postbiotics, let alone an actual microbiota as we propose to do with the present invention. From a practical point of view, the only way to make the use of the in ovo technique for administering the composition according to the invention applicable in the poultry industry would be to combine it with the vaccination for Marek's disease, since it would be impractical, as well as harmful, to carry out two separate administrations.

Marek's Disease is a lymphoproliferative disease that affects chickens and is sustained by an alphaherpesvirus, characterised by the presence of lymphocyte-like mononuclear cell infiltrations localised in various tissues and organs including peripheral nerves, iris, muscles and skin (Calnek., 2001). This disease, which manifests itself with the development of lymphomas, neurological symptoms and immunosuppression, has been the cause of severe economic losses for the industry of the poultry sector, which have been greatly reduced thanks to the development of the vaccination in 1969 that is still commonly administered in ovo, within the amniotic fluid, on the 18th day of incubation (Gimeno, 2008; Reddy et al., 2017).

Vaccination for Marek's disease continues to be administered in ovo as this has been proven to provide the best protection when compared to the subcutaneous injection of the same vaccine at birth (Gimeno et al., 2012a; Gimeno et al., 2012b). In the case of vaccination for Marek's disease, the decision to administer in the amnion on the 18th day of incubation also stems from the demonstration that the amnion turned out to be the most effective site of administration compared to the others (Wakenell et al., 2002). In general, whatever vaccine is used, the possibility of administering substances, such as probiotics, mixed in the vaccine diluent, should be evaluated based on the type of substances used, but especially on the dosages.

The procedure for administering substances in ovo can be performed in different sites and at different ages (e.g. embryonic body, amnion, air chamber), however, the most widely used method is the one patented by Uni et al., (2003), which dictates how the in ovo administration of different substances in the amniotic cavity should be performed. It is well known that the chick in the hatching phase, prior to external pipping, swallows the residue of the amniotic fluid content and thus the substances it contains enter into contact with the gastro-enteric and respiratory tract of the chick itself. The possibility of implementing the administration of different substances, such as probiotics, postbiotics or prebiotics, by mixing them with the diluent for the vaccine, minimises the losses that would have occurred by carrying out a double administration, risking accidental damage to the embryos or contamination with microorganisms present in the hatchery environment.

In the current state of the art, only two studies have combined the administration of probiotics with vaccination for Marek's disease, with positive results (Castaneda et al., 2020; Teague et al., 2017).

The hatching of chicks with an already pre-colonised gut microbiota and a modified pH (tendentially more acidic due to the effect of both the lactic flora, but also due to the large presence of short-chain volatile fatty acids) makes them more resistant to field infections and especially to protozoan colonisation, a prevalent problem in the poultry industry. In addition, a correct development of the gastrointestinal tract is promoted, which is the basis for the reduction of diseases that develop especially in the first weeks of life, allowing a decrease in the use of antibiotics for therapeutic purposes, which is a fundamental aspect in the One Health perspective.

The early maturation of the gastrointestinal tract allows a better use of the feeds, a better food conversion index and thus a faster growth and a higher weight at the slaughter for the same amount of feed consumed (higher digestive efficiency of the chicks pre-colonised in ovo).

### SUBJECT AND SUMMARY OF THE INVENTION

It is therefore an aim of the present invention to provide a composition that stimulates the immune system of the chick, that functions as a probiotic/prebiotic/postbiotic, and that allows to provide the chick with a real complete and balanced gut microbiota.

This aim is achieved by the present invention by means of a composition as defined in Claim 1.

### Definitions

“Lyophilised by spray technique” and the like means processed according to the lyophilisation process described in Italian patent application No. 102021000002000.

“Tyndallised in batch mode” and the like means processed according to the tyndallisation process described in Italian patent application No. 102021000002000.

For the sake of completeness, information on this process as described in the aforementioned patent application is provided below. The process for preparing a composition comprising a dry extract of bovine and ovine rumen content and rabbit caecal content comprises a first stage of tyndallisation in RBI17172-IT 32 batch mode, followed by a second stage of atomisation (spray drying). Preferably, the tyndallisation stage is performed by heating at a temperature comprised between 70-100° C. and for a time comprised between 15 and 30 minutes followed by incubation at room temperature for a period of 24 hours, repeated two or three times. More preferably, the tyndallisation stage includes three heating steps at 70° C. for a maximum of 30 minutes, interspersed with an incubation period of 24 hours. Preferably, the atomisation stage (spray drying) comprises four subsequent steps wherein the starting liquid product is atomised into a spray form, then the droplets generated by the spray are contacted with hot air to allow moisture evaporation and formation of dry solid particles, then the dry solid particles are separated from the air flow and collected. More preferably the atomisation stage (spray drying), the atomisation of the starting liquid product into a spray is performed by means of an atomising device, then the droplets generated by the spray are subjected to contact with heated air in a drying chamber, resulting RBI17172-EN 33 in moisture evaporation and the formation of dry solid particles that are separated from the air flow and collected in a collection device.

The atomisation process (spray drying) is carried out in an atomiser (spray dryer), which comprises a heater for heating the air; an atomiser for atomising the mixture to form micro-particles; a drying chamber where the moist micro-particles enter into contact with hot air that evaporates the water in the micro-particles, generating dry powder; a cyclone separator for collecting the powder; and a fan for discharging the exhausted air.
The spray drying technique allows dry powders with low moisture content to be obtained starting from liquid products such as solutions, emulsions or suspensions. It is possible to obtain a final product with low water activity, guaranteeing microbiological stability, reduced volume and weight, and facilitating storage, transport and marketing. The process comprises four stages. Firstly, the liquid sample is converted into a spray by an atomising device. These small droplets are subjected to contact with heated air in a drying chamber, resulting in the evaporation of moisture and the formation of dry solid particles. Finally, the solid particles are separated from the air flow and collected in a collection device. Sample A was resuspended in ultra-pure water, washed and filtered through a small-mesh sieve to remove suspended solids. The filtered material (Sample C) was resuspended in ultra-pure water (3:1 ratio) and subjected to the spray drying process.
The drying of rumen/caecal juice was performed in a laboratory-scale Bichi Mini Spray Dryer B-290 (Bichi LaboratoriumsTechnik) with a two-nozzle atomiser with an inner diameter of 0.7 mm and a 16-cm simultaneous drying chamber. The two previously prepared solutions (rumen and caecal) (stirring at 40° C.) were inoculated into the chamber via a peristaltic pump at a constant flow rate (9 mL/min). The drying air flow rate was kept at 100% and the compressed air flow rate at 450 L/h. The inlet air temperatures tested were 100 and 120° C. The outlet air temperature cannot be regulated but is the result of the combination of inlet air temperature, feed rate, drying gas flow rate and solids content of the feed. A single cyclone air separator system was used and the dried powders were collected from the base of the cyclone. Two replications were conducted for each experiment. The analysis of the dried powders was performed immediately after spray drying. The drying yield was determined as the percentage ratio between the weight of the total mass of harvested product and the initial amount of solids present in the solution inoculated in the spray dryer. The water activity of the powders was determined using a water activity meter (Aqualab, 4TE, Decagon Devices Inc.) at a constant temperature of 23±1° C. Two readings were taken for each sample.

### DETAILED DESCRIPTION OF PREFERRED EMBODIMENTS OF THE INVENTION

According to the present invention, there is provided a composition comprising dry extract of intestinal content of adult chicken lyophilised by spray technique and tyndallised in batch mode and at least one live bacterial species selected from the group consisting of Enterococcus faecium, Streptococcus thermophilus, Bifidobacterium bifidum, Lactobacillus reuteri, and Lactobacillus acidophilus.

The percentage of supplementation in the dry extract is variable and is determined based on the age, site and route of administration.

The chickens that are the donors of the intestinal content are healthy adult chickens raised without using antibiotics. The intestinal content used for the production of the dry extract is obtained by squeezing the entire intestinal tract, excluding the cloaca. The intestinal packet is taken during slaughter.

Lyophilisation and tyndallisation in batch mode are described in detail in Italian patent application No. 102021000002000.

Tyndallisation preferably takes place by heating at a temperature comprised between 70-100° C. and for a time comprised between 15 and 30 minutes, followed by incubation at room temperature for a period of about 24 hours, repeated two or three times. More preferably, tyndallisation includes three heating steps at 70° C. for a maximum of 30 minutes, interspersed with an incubation period of about 24 hours.

Lyophilisation by spray technique preferably takes place in four subsequent steps wherein the starting liquid product is atomised into a spray form, then the droplets generated by the spray are contacted with heated air for moisture evaporation and formation of the dry solid particles, then the dry solid particles are separated from the air flow and collected.

This treatment allows a dry extract to be yielded that is easy to dilute in any liquid, including the vaccine diluent for Marek's disease. This technique achieves the deactivation of the microorganisms present in the starting material, including coccidia, ensuring the safety of the compound. However, the mixture will provide all the major bacterial, viral and protozoan compounds present in the donors, plus a postbiotic and prebiotic component derived from the microorganisms themselves, from the content of the lysed cells and from bacterial products.

A vaccine for Marek's disease is any vaccine used for the protection against the virus in chickens. The vaccine in question may be composed of viruses belonging to one of the three serotypes MDV-1, MDV-2, MDV-3 (or HVT) individually as a mono-valent vaccine, or in combination as a bi- or tri-valent vaccine. Vaccines can be cell-associated, as for MDV-1 and MDV-2, or lyophilised for HVT-3. In the case recombinant vaccines are used, vaccines comprising HVT associated with Infectious Bursitis, Newcastle Disease, Avian Influenza or Infectious Laryngotracheitis viruses are considered. The vaccine must be approved for in ovo administration.

Diluent means any solution used for the administration of the vaccine for Marek's disease.

The vaccine dosages used are relative to the type of vaccine, age of administration, injection site and reference species. Reference is made to the manufacturer's instructions.

The dosages of the mixture proposed by this patent will also vary based on age of administration and site of inoculation.

The composition according to the present invention can be used for animal food supplementation. As a supplement, the composition is preferably administered in the chickens' food or feed water.

The composition according to the present invention can also be used to stimulate the immune system, with probiotic, prebiotic, and/or postbiotic action.

Preferably, the target species is chicken.

The composition is preferably administered in ovo. Even more preferably inside the amniotic sac.

Even more preferably, the administration takes place from the 12th to the 18th day of incubation of the egg. In a preferred embodiment, the administration takes place on the 18th day of incubation, the age of administration of the vaccination for Marek's disease, as well as the moment for transferring the eggs from the incubation carts to the hatching carts. It is also well established that from the 18th day onwards the chicken embryo begins to have its own functioning immune system, capable of distinguishing self-antigens from not self-antigens.

Preferably, the administration in ovo takes place in an air chamber or in amnion.

The composition is preferably administered in combination with the vaccine for Marek's disease. The site is preferably the amnion in the case of administration of the composition according to the present invention concomitantly with the vaccine for Marek's disease, so that a single injection can be carried out.

The mode of administration includes any automated or manual means. In both modes, it is provided for the egg to be temporarily taken from the incubator machine. Following the candling operation, the position of the air chamber is identified, normally located at the level of the obtuse pole of the egg. Appropriate disinfection of the shell surface with alcohol is carried out. A guide hole is made through which the needle is then inserted, which will allow the compound of the present invention to be deposited either directly at the level of the air chamber or, after passing through the testaceous membrane, into the amnion. This technique can be used indifferently for any dosage of the proposed mixture.

The present invention proposes a fundamental innovation from the point of view of the composition created for in ovo administration. The aim is to achieve with a single injection in ovo, and precisely at the level of the amniotic sac of the embryo, early immunisation against Marek's disease and at the same time an immuno-stimulation that will A) increase the vaccine response; B) fully and effectively develop the chicken's gastroenteric lymphoid system or GALT C) integrate the whole series of bacterial metabolites (short-chain volatile fatty acids; bacterial-derived amino acids and nucleosides; B vitamins and bacterial peptides with immunomodulatory action) that are normally produced by the mature gut microbiota; D) integrate the whole series of antigens derived from inactivated protozoan oocysts that will immunise the chick by reducing the subsequent coccidial colonisation.

### EXAMPLES

### Example 1

The two experiments described below were performed to verify the safety and efficacy of the in ovo administration of the two components of the mixture proposed by this patent, injected individually. A first experiment involved the in ovo administration of three different dosages of the live probiotic mixture. A second experiment, structured like the previous one, was carried out to verify the safety of the in ovo administration of three concentrations of dry extract. The hatching rate following administration was taken into account to verify the possible influence of the technique, or the dosage, on the hatching capacity of the animals. Experiment two also checked the weight of the inoculated subjects at slaughter, the final coccidial load, and some morphological parameters of the gastrointestinal tract (villus height, crypt depth and area of lymphoid tissue or GALT as well as the Bursa of Fabricius) of the chickens derived from the chicks inoculated in amnion with the mixture object of the invention, comparing them with chickens deriving from uninoculated chicks (control group). The results obtained allow to state that all dosages of the dry extract were safe as there was no influence on the hatching rate. Dosages of live probiotic mixture are safe starting from a concentration of 1×10\hat{ }5 CFU. The parameters at slaughter indicate that chickens derived from chicks inoculated in amnion with the mixture object of the invention show a statistically higher Live Weight (LV) than that of the controls, as are statistically significant the differences in morphological parameters (villus length, crypt depth, area of development of the lymphoid tissue GALT) that are much more developed in chicks inoculated in amnion. Finally, chickens derived from chicks inoculated with the mixture show a statistically much lower coccidial load (no. of coccidial oocysts per gram faeces—calculated by the Flotac® method) than control chickens vaccinated after birth with Paracox® anticoccidic vaccine.

### Experiment 1

This example describes the in ovo administration, in amniotic fluid, of the probiotic mixture composed of strains of: Enterococcus faecium, Streptococcus thermophilus, Bifidobacterium bifidum, Lactobacillus reuteri, Lactobacillus acidophilus.

Ross 308 hatching eggs were incubated inside the Fiem model MG 100/150 incubator (Como). On the eighteenth day of incubation 100 Ross-308 fertile eggs were divided into four groups, P1—P2—P3—C composed of 25 eggs each.
Group P1 was administered 0.05 ml of saline containing 1×10\hat{ }6 CFU of live probiotic bacteria.
Group P2 was administered 0.05 ml of saline containing 1×10\hat{ }5 CFU of live probiotic bacteria.
Group P3 was administered 0.05 ml of saline containing 1×10\hat{ }4 CFU of live probiotic bacteria.
Group C was administered only 0.05 ml of solution, without any probiotic addition.

Administration was accomplished by manually injecting the compound into the amniotic fluid.

The hatching rate is slightly reduced in P1 due to the higher concentration of live probiotic bacteria. No reduction in hatchability is observed in P2, P3 and C, a factor which leads the P2 concentration equal to 1×10\hat{ }5 CFU of live probiotic bacteria being considered as safe and effective.

### Experiment 2

This second experiment, structured like the previous one, is aimed at verifying the effects of the administration of the dry extract obtained from the intestinal content of healthy antibiotic-free chickens, lyophilised using a spray technique and then tyndallised according to Italian patent application method No. 102021000002000.

Ross 308 hatching eggs were incubated inside the Fiem model MG 100/150 incubator (Como). On the eighteenth day of incubation 100 Ross 308 fertile eggs were divided into four groups, three groups E1—E2—E3—C composed of 25 eggs each.
Group E1 was administered 0.05 ml of saline containing 850 CFU of dry extract. Group E2 was administered 0.05 ml of saline containing 85 CFU of dry extract.
Group E3 was administered 0.05 ml of saline containing 8.5 CFU of dry extract.
Group C was administered only 0.05 ml of solution, without any probiotic addition.

Administration was accomplished by manually injecting the compound into the amniotic fluid.

No reduction in the hatching rate is observed.

### Example 2

This example describes the effects of the in ovo administration of a vaccine for Marek's disease, the diluent thereof and different concentrations of the mixture proposed by this patent.

On the eighteenth day of incubation 100 Ross 308 fertile eggs were divided into 5 groups.

A first N group, consisting of 10 eggs, was not subjected to any administration procedure.

A second group V, consisting of 10 eggs, was administered only vaccine in an amount of 0.05 ml, suitably mixed in its diluent.

The eggs of the four groups A to D, consisting of 20 eggs respectively, were administered in amnion 0.05 ml of the mixture comprising the vaccine mixed with its diluent, 1×10\hat{ }5 live probiotic bacteria (a mixture of Enterococcus faecium, Streptococcus thermophilus, Bifidobacterium bifidum, Lactobacillus reuteri, Lactobacillus acidophilus containing a total of 200 billion lactic acid bacteria per 1.5 grams of product was administered) and 850, 85, 8.5 and 0.85 CFU respectively derived from the dry extract.

The dry extract of the intestinal content derived from healthy antibiotic-free chickens was obtained with lyophilisation treatment by spray technique and tyndallisation, according to the method of the Italian patent application No. 102021000002000.

The administration procedure was performed manually. After appropriate disinfection of the eggs at the level of the obtuse pole, a guide hole was made using an 18G needle into which a 22 G needle was inserted at a depth of 2.49 cm. The depth of administration is such that the content is deposited directly into the amniotic fluid.

After administration the eggs were transferred to the hatching box and duly completed incubation.

Table 1 shows the hatching rates of the different groups.

TABLE 1

| TREATMENT | HATCHING RATE |
|---|---|
| N - untreated | 10/10 |
| V - vaccine only | 9/10 |
| A - 1 × 10\hat{ }5 probiotic + 850 CFU | 18/20 |
| dry extract | |
| B - 1 × 10\hat{ }5 probiotic + 85 CFU | 18/20 |
| dry extract | |
| C - 1 × 10\hat{ }5 probiotic + 8.5 CFU | 18/20 |
| dry extract | |
| D - 1 × 10\hat{ }5 probiotic + 0.85 CFU | 20/21 |
| dry extract | |

Eggs that did not hatch at the end of the 21-day incubation period were examined to see whether mortality had occurred early or after administration. Only group A and B embryos show late mortality, although there are no evident lesions related to the in ovo administration procedure.

Subsequently, the chicks were housed and reared in separate pens to evaluate the growth performance of the animals. At 28 days of age they were duly slaughtered for human consumption. At the time of slaughter, samples were taken from the organs to perform the morphological evaluation of the different intestinal segments and of the main lymphoid organs.

From the evaluation of the data obtained from the hatching rates, the in ovo administration procedure of the mixture proposed by this patent together with the vaccine for the disease appears to be safe as there is no incidence on the hatching rate except for group A.

The evaluation of the efficacy of the treatment resulted from the set of growth and morphological parameters.

Specifically, the weight of the treated subjects was increased in comparison to the controls (groups N and C) throughout the duration of the experiment. (p<0.05). At the level of intestinal morphology, an increase in the height as well as in the thickness of intestinal villi is observed, indicating a greater absorbent surface following treatment (p<0.005). In addition, there is also a functional increase in the main immune organs, which confirms the immuno-stimulating effect of the treatment (p=0.012).

All the parameters considered contribute to confirming the in ovo treatment according to the present invention, emphasising again its efficacy but above all its safety.

### Advantages

The present invention aims to simultaneously use more than one “biotic” approach, and in particular to use the probiotic/prebiotic/postbiotic approach. The dry extract obtained by lyophilisation and tyndallisation of the intestinal content of healthy adult chickens provides in an inactivated form all the main “antigenic motifs” of bacterial, viral, fungal and protozoal nature that the organism of the chick can acquire after hatching, in live and viable form, from the environment in which it lives and from the contact with the faeces of the parents and siblings. To this typically postbiotic effect (i.e. of presentation of a miscellany of inactivated antigens to the intestinal mucosa, i.e. derived from dead microorganisms, but strongly stimulating the gastrointestinal immune system) in the present invention a probiotic component (consisting of live and viable lactic acid bacteria) and a prebiotic component are added, which is derived from the microorganisms themselves, from the content of the lysed cells and from the bacterial products which, together with the live probiotic bacteria constitute the “compound” which, together with the method of administration, forms part of this invention. The administration of the composition (lyophilised/tyndallised of intestinal content+live lactic bacterial strains), allows to provide the chick with a real complete and balanced gut microbiota.

1. A composition comprising dry extract of intestinal content of adult chicken lyophilised by spray technique and tyndallised in batch mode and at least one live bacterial species selected from the group consisting of Enterococcus faecium, Streptococcus thermophilus, Bifidobacterium bifidum, Lactobacillus reuteri, and Lactobacillus acidophilus.

2. The composition according to claim 1, wherein the tyndallisation is performed by heating at a temperature comprised between 70-100° C. and for a time comprised between 15-30 minutes followed by incubation at room temperature for a period of about 24 hours, repeated two or three times.

3. The composition according to claim 1, wherein lyophilisation by spray technique takes place in four subsequent steps wherein the starting liquid product is atomised into a spray form, then the droplets generated by the spray are contacted with heated air for moisture evaporation and formation of dry solid particles, then the dry solid particles are separated from the air flow and collected.

4. A use of the composition according to claim 1 for veterinary food supplement.

5. The composition according to claim 1 for use for stimulating the immune system, with probiotic, prebiotic and/or postbiotic action.

6. The composition for use according to claim 5, wherein the target species is chicken.

7. The composition for use according to claim 5, wherein the composition is administered in ovo.

8. The composition for use according to claim 7, wherein the administration is performed from the 12th to the 18th day of incubation in the egg.

9. The composition for use according to claim 7, wherein the composition is administered in combination with the vaccine for Marek's disease.
